(19) United States
(12) Patent Application Publication
(10) Pub. No.: US 2025/0283808 A1
(43) Pub. Date: Sep. 11, 2025

(54) GAS-MEASURING DEVICE WITH A SENSOR AND WITH MULTIPLE GAS INLETS AS WELL AS ACTUATION ARRANGEMENT, OPERATING METHOD, AND MEASURING METHOD FOR SUCH A GAS-MEASURING DEVICE

(71) Applicant: Dragerr Safety AG & Co . KGaA, Lubeck (DE)

(72) Inventors: Rüdiger Weich, Lubeck (DE); Sven Eric Schmaljohann, Lubeck (DE); Marcel Hirzel, Lubeck (DE)

(21) Appl. No.: 19/059,440

(22) Filed: Feb. 21, 2025

(30) Foreign Application Priority Data
Mar. 5, 2024 (DE) 102024106251.7

(51) Int. Cl. G01N 21/3504; G01N 33/00

(52) U.S. Cl. CPC G01N 21/3504; G01N 33/0073

(57) ABSTRACT

The disclosure relates to an actuation arrangement for actuating a gas-measuring device, to an operating method for operating a gas-measuring device, to a gas-measuring device and to a measuring method using a gas-measuring device. The gas-measuring device comprises two gas inlet sets, each with a gas inlet. A measuring chamber takes up a gas sample. A sensor measures the concentration of a target gas in the held gas sample. A fluid-conveying unit conveys gas from the gas inlets to the measuring chamber. During operation in a detection mode, a fluid connection is established between each gas inlet and the measuring chamber. From each gas inlet respective gas is conveyed into the measuring chamber. During operation in a differentiation mode, a respective measuring phase is carried out for each gas inlet set. During the measuring phase for a gas inlet set, the gas inlets of this gas inlet set are connected to the measuring chamber and any other gas inlet is disconnected from the measuring chamber. A gas from the gas inlets of the gas inlet set is conveyed into the measuring chamber.

### CROSS-REFERENCE TO RELATED APPLICATIONS

This application claims the priority of German Patent Application No. 102024106251.7, filed on Mar. 5, 2024, and titled “GAS-MEASURING DEVICE WITH A SENSOR AND WITH MULTIPLE GAS INLETS AS WELL AS ACTUATION ARRANGEMENT, OPERATING METHOD, AND MEASURING METHOD FOR SUCH A GAS-MEASURING DEVICE”, which is hereby incorporated by reference in its entirety for all nonlimiting purposes.

The disclosure relates to an actuation arrangement for actuating a gas-measuring device, to an operating method for operating a gas-measuring device, to a gas-measuring device, and to a measuring method using such a gas-measuring device.

Gas-measuring devices that comprise a measuring chamber, a sensor, a fluid-conveying unit, and a gas inlet have become known. At least temporarily a fluid connection is established between the gas inlet and the measuring chamber. A fluid-conveying unit conveys a gas sample from the gas inlet through the fluid connection into the measuring chamber. The sensor measures the concentration of at least one target gas in the gas sample located in the measuring chamber.

Such a gas-measuring device is, for example, installed or set up at a location and monitors a spatial region for at least one target gas to be detected. It is also possible for a user to carry such a gas-measuring device while the user is in a spatial region in which the target gas or at least one target gas to be detected occurs or can occur. The target gas is, for example, a flammable or toxic gas or a gas that is otherwise harmful to humans. The target gas may instead be oxygen or an anesthetic. The gas-measuring device according to the disclosure can also be used to detect at least one of the target gases just mentioned.

The disclosure is based on the object of providing an actuation arrangement for actuating a gas-measuring device and an operating method for operating a gas-measuring device, wherein the gas-measuring device comprises in each case multiple gas inlets for one gas sample each, and wherein by actuation and operation of the gas-measuring device at least one target gas is to be detected with greater reliability than by known actuation arrangements and operating methods. Furthermore, the disclosure is based on the object of providing a gas-measuring device with multiple gas inlets and a measuring method using such a gas-measuring device, wherein the gas-measuring device and the measuring method are able to detect at least one target gas with greater reliability than known gas-measuring devices and measuring methods.

The object is achieved by an actuation arrangement having the features of claim 1, by a gas-measuring device having the features of claim 8, by an operating method having the features of claim 13 and/or by a measuring method having the features of claim 15. Advantageous embodiments are described in the dependent claims. Advantageous embodiments of the actuation arrangement according to the disclosure are, where applicable, also advantageous embodiments of the gas-measuring device according to the disclosure, the operating method according to the disclosure, and the measuring method according to the disclosure, and vice versa.

Remark: The sequence in which in a claim the steps of a procedure are specified does not necessarily specify a temporal sequence in which these steps are actually executed (carried out).

The gas-measuring device according to the disclosure comprises a measuring chamber and a sensor. The measuring chamber is able to take up (hold) a gas sample. This gas sample comes from a spatial region to be monitored and contains at least one target gas to be detected. It is possible that the spatial region to be monitored and therefore also the gas sample simultaneously contain at least two target gases to be detected. The sensor is able to determine the concentration of the target gas or of at least one target gas to be detected in the gas sample while the gas sample is in the measuring chamber. Optionally, the sensor is able to determine each concentration of at least two target gases to be detected or else the sum of the concentrations of several (multiple) target gases.

In the following, the term “the target gas” is used for short, even though multiple target gases may be present at the same time. The target gas may be a gas harmful to humans, such as a flammable or toxic or otherwise harmful gas, such as methane or carbon monoxide. The target gas can also be a gas that is essential for human life, such as oxygen. The target gas can also be, for example, carbon dioxide or an anesthetic. The gas-measuring device can be used to detect the undesirable event in which the concentration of the target gas is outside a specified target value range. In particular, the event can be detected in which a harmful gas is present at a too high concentration or oxygen is present at a too low concentration.

Note: The wording is used that a sensor is able to measure a physical variable, in the present case the concentration of a target gas in a gas sample. This wording means that the sensor is able to measure the physical variable directly or that it is able to measure at least one other variable, wherein the other variable or the combination of the other variables correlates with the variable to be measured and is therefore an indicator for the physical variable to be measured. The measurement provides at least one value for the physical variable sought.

In addition, the gas-measuring device comprises at least two gas inlet sets. Each gas inlet set comprises at least one respective gas inlet. Each gas inlet belongs to exactly one gas inlet set. Therefore, the gas-measuring device comprises at least two gas inlets. It is possible for the gas-measuring device to comprise an additional gas inlet, wherein the additional gas inlet does not belong to a gas inlet set. For example, the additional gas inlet is used to suck in a gas for a reference measurement or a gas for purging or cleaning the measuring chamber. In the following the term “the gas inlets of the gas-measuring device” is used. This term refers to the gas inlets which belong to a gas inlet set.

Each gas inlet defines a measuring position. Each gas inlet and thus each measuring position is spatially remote (spaced, distanced) from the other or any other measuring position.

A fluid connection arrangement of the gas-measuring device connects each gas inlet belonging to a gas inlet set to the measuring chamber by a respective fluid connection, at least temporarily. A fluid connection arrangement is understood to mean a component with multiple inlets and at least one outlet, wherein the component establishes a respective fluid connection between each inlet and each outlet. A Y-piece is an example of a component with two inlets and one outlet.

Furthermore, the gas-measuring device comprises at least one fluid-conveying unit, preferably with at least two fluid-conveying units arranged in parallel. A “fluid-conveying unit” is understood to mean a component that is able to convey a fluid and has an inlet and an outlet for the fluid. A fluid-conveying unit generally has a negative pressure side and a positive pressure side. A pump, a piston-cylinder unit, and a blower are examples of a fluid-conveying unit.

The or each fluid-conveying unit is configured to carry out the following process: The fluid-conveying unit conveys a quantity of a gas from at least one gas inlet through the fluid connection arrangement to the measuring chamber. Thereby the gas quantity is conveyed into the measuring chamber. To each gas inlet set a respective fluid-conveying unit is assigned. It is possible that to each gas inlet set a different fluid-conveying unit is assigned. It is also possible for at least two different gas inlet sets to be assigned the same fluid-conveying unit.

If a gas inlet set comprises at least two gas inlets, then at any given time either gas is sucked-in through every gas inlet of this gas inlet set or through no gas inlet of this gas inlet set. The situation in which gas is sucked-in through one gas inlet of the gas inlet set but not through another gas inlet of the same gas inlet set generally cannot occur.

“Assignment of a fluid-conveying unit to a gas inlet set” means in particular that a unique identifier for each gas inlet set and each fluid-guiding unit as well as the information which fluid-conveying unit is assigned to which gas inlet set are stored in a data storage device or a program. These identifiers and assignments are used while the gas-measuring device is in use and, for example, a control unit executes a program and/or accesses a data storage device.

The gas-measuring device can be operated either in a detection mode or in a differentiation mode. The actuation arrangement according to the disclosure is configured to actuate the gas-measuring device such that the gas-measuring device is operated in the detection mode or in the differentiation mode depending on the actuation. The operating method according to the disclosure causes the gas-measuring device to be operated at times in the detection mode and at times in the differentiation mode. Of course, in addition to these two modes, an idle state is also possible in which the gas-measuring device is switched off and, for example, cleaned.

In the detection mode, the gas-measuring device is able to detect whether or not at at least two different measuring positions the same target gas has occurred with a target gas concentration outside a given concentration range, in particular with a high target gas concentration. In the differentiation mode, the gas-measuring device is able to distinguish (identify) the measuring position at which this target gas concentration outside the concentration range has occurred. These two modes are explained in more detail below.

The gas-measuring device according to the disclosure is configured to function as follows during operation in the detection mode, and the actuation arrangement according to the disclosure and the operating method according to the disclosure bring about the following functionality during operation in the detection mode:
A respective fluid connection is established between each gas inlet, i.e., between each measuring position, on one side, and the measuring chamber on the other side. If this fluid connection has not yet been established, it is established. This fluid connection leads through the fluid connection arrangement.
The or at least one fluid-conveying unit conveys from the or each gas inlet belonging to a gas inlet set a respective quantity of a gas into the measuring chamber. The conveyed gas flows through the fluid connection arrangement to and into the measuring chamber.
A gas sample is formed in the measuring chamber. This gas sample is created by mixing the gases from all the gas inlets of the at least two gas inlet sets.
For the or each target gas, the sensor measures the concentration of the target gas in the gas sample which gas sample has been or is created by mixing in the measuring chamber.

The gas-measuring device according to the disclosure is configured to carry out during operation in the differentiation mode for each gas inlet set at least one respective measuring phase. Optionally, the gas-measuring device is able to carry out at least two measuring phases for the same gas inlet set. The measuring method according to the disclosure comprises at least one such respective measuring phase.

When the gas-measuring device carries out a measuring phase for a gas inlet set x, the following states are established, and the following steps are carried out:
A fluid connection is established between the or each gas inlet of this gas inlet set x and the measuring chamber. If such a fluid connection has not yet been established, it is established. This fluid connection leads through the fluid connection arrangement.
Any other gas inlet, i.e., a gas inlet that does not belong to this gas inlet set but to the other or another gas inlet set y #x, is disconnected from the measuring chamber. This disconnection prevents a flow of gas from this other gas inlet to the measuring chamber.
The or a fluid-conveying unit conveys a quantity of a gas into the measuring chamber. This gas comes (stems) from the gas inlet or gas inlets of the gas inlet set x for which the measuring phase is carried out.
As a result, a gas sample comprising gas from the or each gas inlet of the gas inlet set x for which the measuring phase is carried out is formed in the measuring chamber. Ideally, at least at the end of the measuring phase, this gas sample consists exclusively of gas from the gas inlets of the gas inlet set x and is free of gas from a gas inlet of another gas inlet set y #x.
The sensor measures the concentration of the or each target gas to be detected in the gas sample, which has been created in the measuring chamber at the latest at the end of the measuring phase for this gas inlet set, optionally the sum of several target gas concentrations.

In the measuring phase for a gas inlet set x, a gas sample that comes from the measuring position or positions of this gas inlet set x is thus examined.

After completion of a measuring phase for a gas inlet set x, the concentration at which the target gas occurs at the gas inlet or gas inlets of this gas inlet set x is known. If the gas inlet set x consists of exactly one gas inlet, the concentration at which the or each target gas to be detected occurs at this measuring position is known. If the gas inlet set x comprises at least two gas inlets, in many cases the design of the gas-measuring device determines the mixing ratio with which the gases from these gas inlets are mixed in the gas sample. This effect is achieved because the same fluid-conveying unit is assigned to the gas inlet set x and because the design of the gas-measuring device and thus the cross-sectional areas and pneumatic resistances of the gas inlets are known. It is also possible to use a predefined standard mixing ratio.

Thanks to the disclosure, at least two spaced-apart measuring positions can be monitored for the presence of at least one target gas to be detected. The concentration of the same target gas may differ from measuring position to measuring position, in particular if one measuring position is positioned in a container and another measuring position is outdoors or if the two measuring positions are in two different containers. The disclosure does not require that a separate respective gas-measuring device is used for each measuring position. Rather, the same measuring chamber and the same sensor can be used to monitor the at least two spaced-apart measuring positions.

In many cases, the disclosure makes it possible to quickly detect the undesirable event in which the concentration of the target gas is outside a given target concentration value range and also to detect, at least approximately, the measuring position at which this undesirable event occurred. Therefore, the disclosure on the one hand makes it possible to detect a situation that may be dangerous for humans and on the other hand makes it easier in many cases to locate the cause (origin) of this danger.

The gas-measuring device according to the disclosure can be operated either in the detection mode or in the differentiation mode. The actuation arrangement according to the disclosure and the operating method according to the disclosure bring about (cause) these two modes. Operation in the detection mode has in particular the advantage that all gas inlet sets are simultaneously monitored for the target gas. Operation in the differentiation mode makes it possible to select the gas inlet set from which a high target gas concentration is coming. Advantages of this design, in comparison with a design in which a gas-measuring device has several gas inlets but can only carry out one of these two modes, are explained below.
If the gas-measuring device could operate only in the detection mode, it would be able to detect an undesirable target gas concentration at at least one gas inlet. However, the gas-measuring device would not be able to locate (identify) the gas inlet set to which this gas inlet belongs. Therefore, the gas-measuring device would in many cases be unable to provide any indication as to where the cause of this undesirable target gas concentration lies.
If the gas-measuring device could operate only in the differentiation mode, it would have to carry out in each case one measuring phase for each gas inlet set. Only then could each measuring position be monitored for the presence of a target gas with an undesirable concentration. A measuring phase inevitably requires a certain amount of time. In particular, time is inevitably required to convey gas from the gas inlets of the gas inlet set to the measuring chamber during the measuring phase. In some cases, the process in which the sensor measures the target gas concentration also requires a certain amount of time. During this time, the undesirable event in which an undesirable target gas concentration occurs at another measuring position remains undetected. This undesirable event is only detected later when the measuring phase for the corresponding gas inlet set is carried out.

The disclosure allows, for example, the following application: Humans (workers) are to carry out certain tasks in two containers or rooms. To carry out these tasks, one respective human must be in each of the two containers.

Therefore, these containers or rooms must be monitored for harmful target gases. The gas inlets of one gas inlet set are located in a first container or room, and the gas inlets of the second gas inlet set are located in a second container or room. On the one hand, the disclosure generally ensures that the occurrence of a harmful target gas is detected quickly. On the other hand information is provided as to the container or room of these two containers or rooms in which this harmful target gas has escaped and occurs.

In one embodiment, the gas-measuring device comprises the actuation arrangement according to the disclosure. In particular, the actuation arrangement comprises a control unit of the gas-measuring device, and the control unit is configured to put the gas-measuring device either into the detection mode or into the differentiation mode. In another embodiment, the actuation arrangement is spatially remote from the gas-measuring device. A data connection is established between the actuation arrangement and the gas-measuring device, at least temporarily. The actuation arrangement is able to remotely actuate the gas-measuring device and put it into either the detection mode or the differentiation mode.

In one embodiment, the actuation arrangement or the gas-measuring device itself are able to cause the gas-measuring device to be operated either in the detection mode or in the differentiation mode. For doing so, an external specification is evaluated. The specification comes, for example, from a user or from a higher-level controller.

In another embodiment, the actuation arrangement or the gas-measuring device itself is able to cause the gas-measuring device to be operated either in the detection mode or in the differentiation mode. Preferably, the gas-measuring device is operated in the detection mode or in the differentiation mode depending on the or one measured target gas concentration. This target gas concentration is measured by the sensor of the gas-measuring device and relates to a gas sample in the measuring chamber.

Particularly preferably, the gas-measuring device is operated in the detection mode as long as the respective measured concentration of the or each target gas is within a target value range. This target value range can vary from target gas to target gas. The event that the measured concentration of the or a target gas is outside the target value range is undesirable and often dangerous or harmful. Therefore, the detection of this undesired event causes the gas-measuring device to be switched to the differentiation mode by the actuation arrangement or to switch itself automatically to the differentiation mode and, after switching, to carry out a respective measuring phase for each gas inlet set. Preferably, the gas-measuring device is switched back to the detection mode or switches itself to the detection mode when the or each target gas concentration measured in the differentiation mode is again within the respective target value range.

According to the disclosure, the gas-measuring device can be operated either in the detection mode or in the differentiation mode, in particular due to a corresponding actuation by the actuation arrangement. Different embodiments are possible to bring about these different modes.

In one embodiment, the comprises at least two fluid-conveying units, particularly preferably one respective fluid-conveying unit per gas inlet set. To each gas inlet set a respective fluid-conveying unit is assigned. Particularly preferably, two different fluid-conveying units are assigned to two different gas inlet sets. According to this embodiment, each fluid-conveying unit can be switched on and off independently of the or any other fluid-conveying unit. The actuation arrangement is able to actuate each fluid unit and, through the actuation, switch the fluid-conveying unit on and off again. If a fluid-conveying unit is switched on, it conveys gas from the gas inlet or gas inlets of that gas inlet set to which this fluid-conveying unit is assigned through the fluid connection arrangement to the measuring chamber.

By appropriate actuation, the actuation arrangement is able to bring about the following effects:
During operation in the detection mode, each fluid-conveying unit is switched on. This causes gas to be conveyed from all the gas inlets of the gas inlet sets to the measuring chamber. In other words: As a result of all fluid-conveying units being switched on, the gas-measuring device is put into the detection mode.
In the differentiation mode, a measuring phase for a gas inlet set x is established and carried out as follows: The fluid-conveying unit assigned to this gas inlet set x is switched on. The or any other fluid-conveying unit is switched off. The switched-on fluid-conveying unit conveys gas from the or every gas inlet of the gas inlet set x to the measuring chamber.

The following effect generally occurs during a measuring phase for a gas inlet set x: The switched on fluid-conveying unit causes a positive pressure to occur in the measuring chamber. This positive pressure prevents gas from a gas inlet of another gas inlet set y #x from entering the measuring chamber, even if the measuring chamber is in a fluid connection with the environment.

In a further development of this embodiment, the gas-measuring device comprises a third gas inlet set and a third fluid-conveying unit. By switching on the third fluid-conveying unit, the measuring phase for the third gas inlet set is carried out.

The embodiment just described with the fluid-conveying units that can be switched on and off can be realized in conjunction with at least one changeover valve. However, the embodiment eliminates the need to use a changeover valve to put the gas-measuring device either in the detection mode or in the differentiation mode.

According to the embodiment described below, however, the gas-measuring device comprises a controllable changeover valve. The changeover valve comprises at least two inputs and at least one output, in one embodiment at least two outputs.

In the embodiment with the changeover valve, the fluid connection arrangement of the gas-measuring device comprises a first input fluid-guiding unit, a second input fluid-guiding unit, and an output fluid-guiding unit. Each of these three fluid-guiding units establishes a respective fluid connection. The first input fluid-guiding unit establishes a fluid connection between the or each gas inlet of a first gas inlet set and the first input of the changeover valve. The second input fluid-guiding unit establishes a fluid connection between the or each gas inlet of a second gas inlet set and the second input. The second gas inlet set is different from the first gas inlet set. The two input fluid-guiding units are preferably not fluid-connected to one another but are fluid-tightly disconnected from one another. The output fluid-guiding unit establishes a respective fluid connection between the or each output of the changeover valve on one side and the measuring chamber on the other side. It is possible for the changeover valve to comprise two outputs and for the output fluid-guiding unit to establish two different fluid connections, namely in each case one between an output and the measuring chamber, wherein these two fluid connections are preferably disconnected from one another.

The actuation arrangement is able to actuate (control) the changeover valve. Depending on the actuation, the changeover valve is moved in to a first position, a second position, or a third position and preferably held in this position until the changeover valve is actuated again. In the first position, the changeover valve establishes a fluid connection between the first input and the or at least one output, in the case of several outputs preferably a fluid connection between the first output and each of the at least two outputs of the changeover valve. In the first position, the changeover valve disconnects the second input from the or each output. In the second position, the changeover valve establishes a fluid connection between the second input and the or at least one output and disconnects the first input from the or each output. In the third position, the changeover valve establishes a fluid connection both between the first input and the second input on one side and the or at least one output on the other side.

Depending on the position of the changeover valve, the gas-measuring device is operated either in the detection mode or in the differentiation mode. With other words: The current position of the changeover valve determines in which mode the gas measuring device is currently operated. When the changeover valve is in the third position, the gas-measuring device is in the detection mode. When the changeover valve is in the first or the second position, the gas-measuring device is in the differentiation mode. When the gas-measuring device carries out the measuring phase for the first gas inlet set, the changeover valve is in the first position. The gas-measuring device carries out the measuring phase for the second gas inlet set with the changeover valve in the second position.

The embodiment with the changeover valve that can be moved in the three positions can be combined with the embodiment just described in which two fluid-conveying units are used, wherein each fluid-conveying unit can be switched on and off. However, the embodiment with the changeover valve requires only one fluid-conveying unit and does not necessarily require that this one fluid-conveying unit can be switched on and off during operation.

In many cases, it is sufficient to move the changeover valve to the third, first or second position to cause the gas-measuring device to be in the detection mode or in the measuring phase for the first gas inlet set or in the measuring phase for the second gas inlet set. It is possible, but thanks to the changeover valve not necessary to switch the or one fluid-conveying unit on or off during operation. Rather, it is possible for the or each fluid-conveying unit to remain switched on during operation.

In a further development, the gas-measuring device comprises a third gas inlet set. A third input of the changeover valve is fluid-connected to the gas inlet or gas inlets of the third gas inlet set. In both the first and second positions, the changeover valve disconnects the third input from the or each output. In the third position, the changeover valve additionally connects the third input to the or at least one output. When the gas-measuring device carries out the measuring phase for the third gas inlet set, the changeover valve is in a fourth position. In this fourth position, the changeover valve connects the third input to the or at least one output and disconnects both the first and second inputs from the or each output.

So far, the term “the changeover valve” has been used. In particular, the embodiment in which the changeover valve comprises three inputs can also be realized with multiple individual valves. The term “the changeover valve” also includes this implementation.

According to the embodiment just described, the output fluid-guiding unit connects the or each output of the changeover valve to the measuring chamber. In a further development of this embodiment, the changeover valve comprises two outputs. The output fluid-guiding unit comprises a first fluid-guiding unit and a second fluid-guiding unit. The first fluid-guiding unit establishes a fluid connection between the first output of the changeover valve and the measuring chamber. The second fluid-guiding unit establishes a fluid connection between the second output and the measuring chamber. Preferably, these two fluid-guiding units are fluid-tightly disconnected from one another.

According to the disclosure, the gas-measuring device comprises at least one fluid-conveying unit. In one embodiment, the gas-measuring device comprises two fluid-conveying units. The first fluid-conveying unit is able to convey a gas through the first fluid-guiding unit to the measuring chamber, i.e., from the first output of the changeover valve to the measuring chamber. The second fluid-conveying unit is able to convey a gas through the second fluid-guiding unit to the measuring chamber, i.e., from the second output of the changeover valve to the measuring chamber. According to the embodiment with the two outputs of the changeover valve and the two fluid-conveying units, the changeover valve is preferably configured as follows:

In the first position, the changeover valve establishes a fluid connection between the first input and the first output and disconnects each input from the second output. In the second position, the changeover valve establishes a fluid connection between the second input and the first output and disconnects each input from the second output. In the third position, the changeover valve establishes a fluid connection between the first input and the second input on one side and the second output on the other side and disconnects each input from the first output.

This embodiment can be combined with an embodiment in which the fluid-guiding units are switched on and off during operation, preferably independently of one another. However, the embodiment eliminates the need to switch a fluid-guiding unit on and off during operation. Rather, the mode and, in the differentiation mode, the measuring phase are determined by the position of the changeover valve.

The embodiment makes it possible to use different fluid-conveying units. The first fluid-conveying unit is used in the differentiation mode during the measuring phases, the second fluid-conveying unit is used in the detection mode. The embodiment makes it possible to adapt these two fluid-guiding units to different requirements, in particular the following:

In the detection mode, the measuring positions at the gas inlets are to be continuously monitored for the presence of at least one target gas. The gas-measuring device, on the other hand, is switched into the differentiation mode and operated in the differentiation mode if a target gas has been detected in the detection mode. This detection is generally an undesirable event. Therefore, it is generally desired to be able to find the cause of this undesirable event quickly and to rectify the undesirable event. For example, a leak from which a flammable or otherwise harmful target gas is escaping should be quickly detected and sealed.

Therefore, in the differentiation mode, it is important to quickly convey from each gas inlet set successively a respective gas sample into the measuring chamber and to examine it there. In contrast, the gas-measuring device is generally operated predominantly in the detection mode during use, usually at least 90% of the time it is in use. In particular when the gas-measuring device is not connected to a stationary power supply network during use and therefore comprises its own power supply unit, it is important that it consumes little electrical energy.

For the reasons just mentioned, it is often advantageous to consume relatively little electrical energy in the detection mode but to convey a gas sample rapidly into the measuring chamber in the differentiation mode, even if a relatively large amount of electrical energy is consumed in the differentiation mode. This effect can be realized thanks to the embodiment just described using two different fluid-guiding units. The first fluid-conveying unit is used in the differentiation mode, the second fluid-conveying unit in the detection mode.

In one implementation, the first fluid-conveying unit consumes more electrical energy than the second fluid-conveying unit and as a result is able to achieve a higher volume flow and/or mass flow of a gas to the measuring chamber. Preferably, the first fluid-conveying unit is switched off in the detection mode, the second fluid-conveying unit is switched off in the differentiation mode. This saves electrical energy.

In an exemplary embodiment, a housing of the gas-measuring device accommodates at least the measuring chamber and the sensor, preferably also a control unit. Preferably, a display unit, on which measured target gas concentrations are visually output, is arranged on a wall of the housing. Optionally, the gas-measuring device comprises an alarm unit, which indicates an alarm visually and/or acoustically and/or haptically (by vibrations) when a high target gas concentration occurs.

Preferably, the gas-measuring device is able to generate a message, wherein this message contains, for each target gas and for each measuring position, the information as to the concentration at which this target gas occurs at this measuring position. This message is output in at least one form perceivable by a human, namely on an output unit of the gas-measuring device itself and/or on an output unit of a spatially remote receiver.

It is possible that there is at least one gas inlet in the housing. In an exemplary embodiment, however, the or at least one, preferably each, gas inlet of a gas inlet set is located outside the housing and is connected to a respective opening in the housing by a fluid-guiding unit, preferably a flexible fluid-guiding unit. The or each gas inlet of a first gas inlet set can be positioned freely relative to the or each gas inlet of the or each further gas inlet set, of course only within a range predefined and limited by the fluid connection arrangement. Accordingly, the or each gas inlet of a second gas inlet set can be positioned freely.

This embodiment increases the flexibility of the gas-measuring device, in comparison with a design in which the or each gas inlet is located in or on a housing of the gas-measuring device, for example in each case at the free end of a tube. A gas inlet in or on the housing means that the gas inlet is fixed relative to the housing and is therefore immovable. In particular, thanks to the fluid-guiding unit outside the housing, it is possible to check two containers simultaneously to determine whether or not a target gas occurs at a high target gas concentration inside.

In the following, the disclosure is described on the basis of an exemplary embodiment. In the drawings:

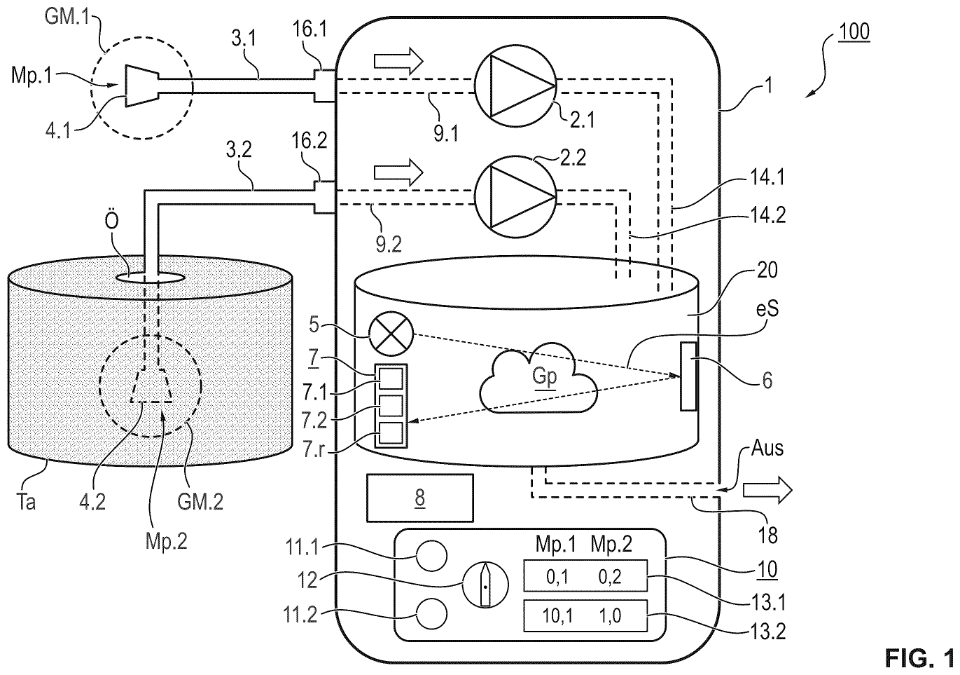

FIG. 1 schematically shows a first embodiment of the gas-measuring device according to the disclosure, in which in the detection mode both pumps convey gas;

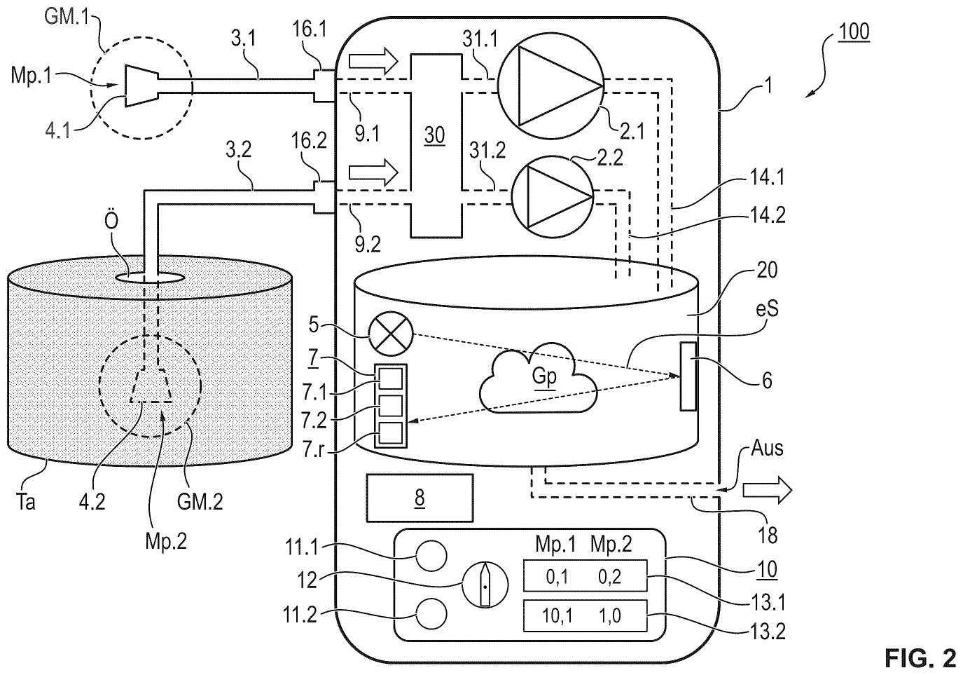

FIG. 2 schematically shows a second embodiment of the gas-measuring device according to the disclosure, in which in the detection mode only one pump conveys gas;

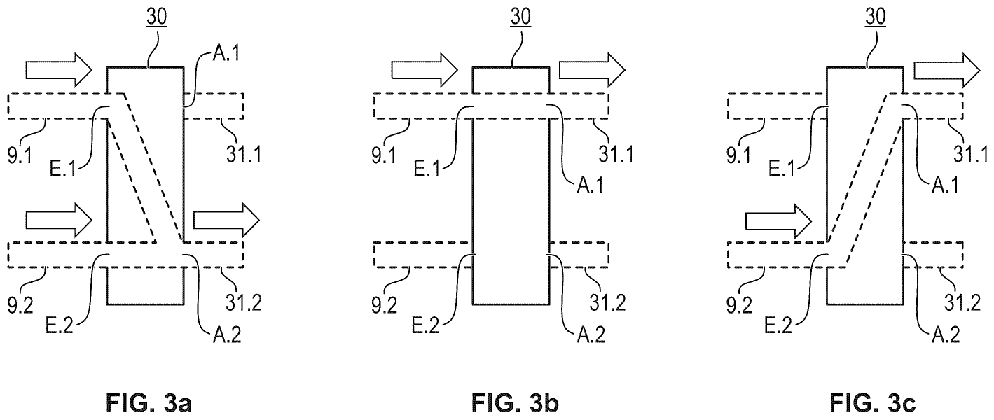

FIGS. 3a-3c show three different positions according to the first implementation of the changeover valve according to the second embodiment;

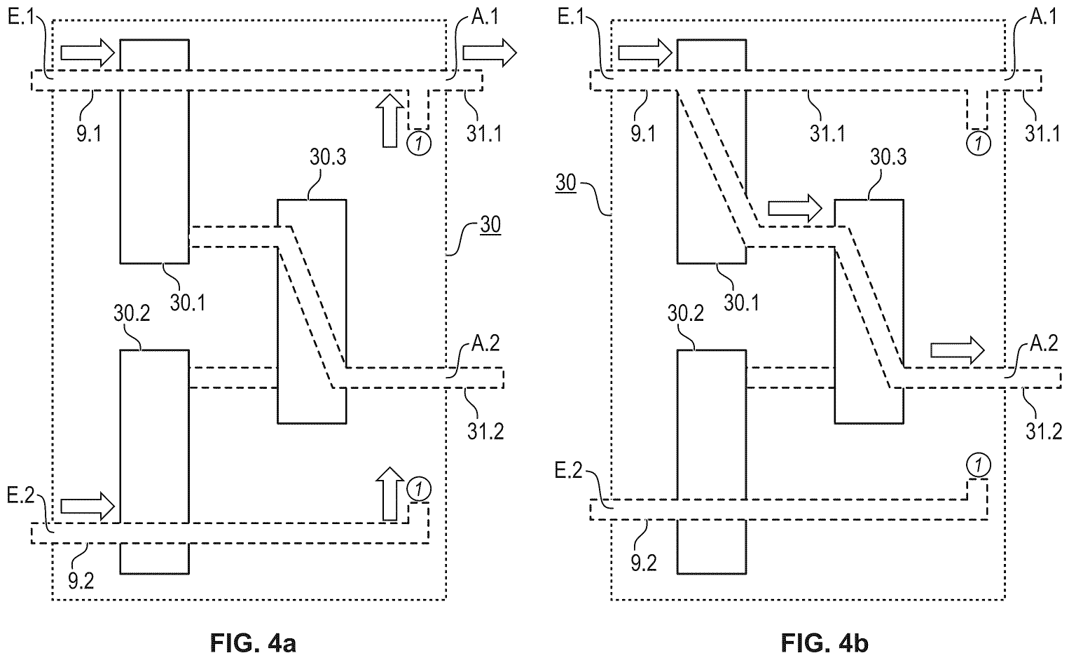

FIGS. 4a-4b show two of the three different positions according to the second implementation of the changeover valve according to the second embodiment;

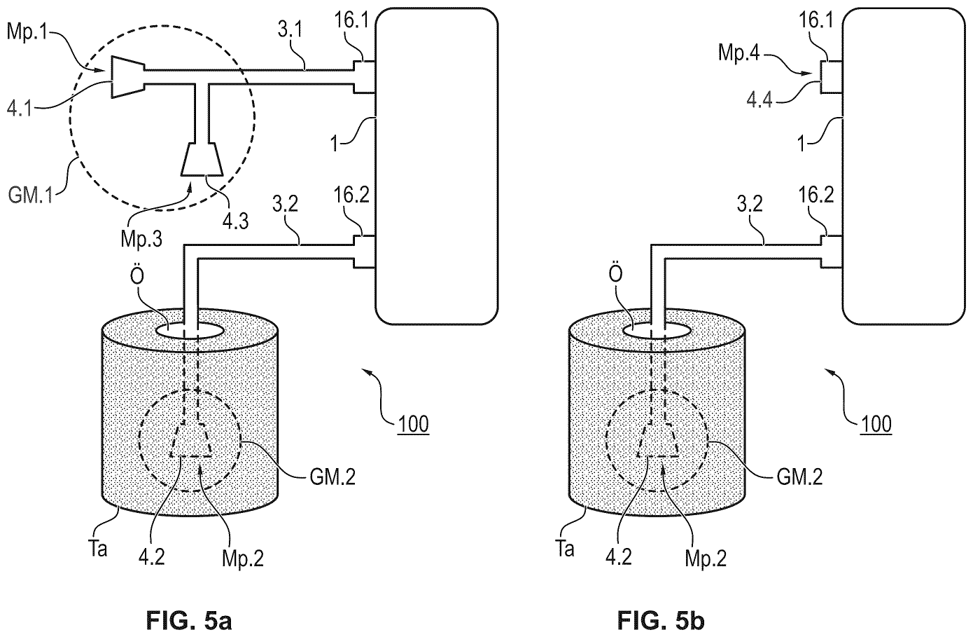

FIGS. 5a-5b show two further example embodiments of the gas-measuring device according to the disclosure;

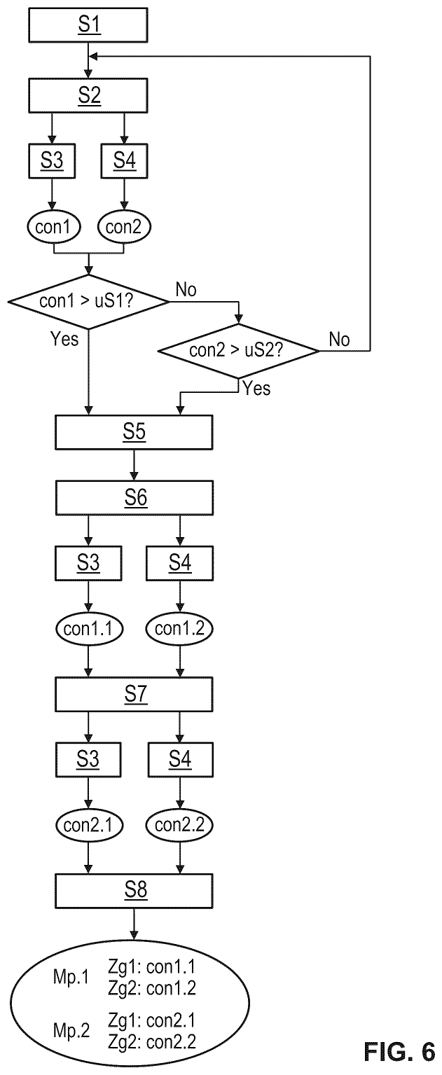

FIG. 6 shows a flow chart for an exemplary embodiment of the method according to the disclosure.

FIG. 1 schematically shows a first embodiment of the gas-measuring device according to the disclosure. The gas-measuring device 100 comprises a housing 1. Inside the housing 1, a measuring chamber 20 is arranged, which chamber has in one implementation the shape of a cylinder. Of course, other geometric shapes of the measuring chamber 20 are also possible. The measuring chamber 20 receives a gas sample Gp to be examined. This gas sample Gp can contain at least one target gas, and this target gas is to be detected. The or a target gas is, for example, flammable or toxic or otherwise harmful to humans. The target gas can also be, for example, an anesthetic or oxygen.

The gas-measuring device 100 comprises a sensor. Various principles according to which a sensor of the gas-measuring device 100 can operate are possible. In the exemplary embodiment shown, the sensor is designed as a photoelectric sensor.

At one wall of the measuring chamber 20 a radiation source 5 and a detector component 7 are located on a circuit board. On the opposite wall there is a mirror 6, or the wall itself acts as the mirror 6. The radiation source 5 emits electromagnetic radiation eS into the measuring chamber 20, preferably radiation in the infrared range. The electromagnetic radiation eS penetrates the gas sample Gp in the measuring chamber 20 a first time, is reflected by the mirror 6, penetrates the gas sample Gp a second time, and strikes (impinges onto) the detector component 7. Thanks to mirror 6, the optical path is doubled.

In one embodiment, the detector component 7 comprises a first measuring detector 7.1, a second measuring detector 7.2, and a reference detector 7.r. In front of each detector 7.1, 7.2, 7.r there is a respective wavelength filter that only allows electromagnetic radiation eS within a certain wavelength range to pass through (penetrate). The wavelength filter in front of the first measuring detector 7.1 allows electromagnetic radiation eS within a first wavelength range to pass through, wherein a first target gas to be detected attenuates electromagnetic radiation eS within this first wavelength range. Accordingly, the wavelength filter in front of the second measuring detector 7.2 allows electromagnetic radiation eS within a second wavelength range to pass through, wherein a second target gas attenuates electromagnetic radiation eS within the second wavelength range. The two wavelength ranges are different from one another and can overlap or be disjoint. The wavelength filter in front of the reference detector 7.r attenuates radiation within a reference wavelength range. Each detector 7.1, 7.2, 7.r generates a respective signal that correlates with the intensity of incident electromagnetic radiation eS.

Optionally, a wall (not shown) separates the radiation source 5 and the detector component 7 fluid-tightly from a gas sample Gp inside the measuring chamber 20. In one embodiment, the entire wall is permeable to the electromagnetic radiation eS. In another embodiment, at least two windows that are permeable to the electromagnetic radiation eS are inserted into the wall, and the rest of the wall is impermeable to radiation. The optional wavelength filters can also be designed as windows in this wall or form the windows in the wall.

A signal-processing control unit 8 inside the housing 1 receives a signal in each case from the first measuring detector 7.1, from the second measuring detector 7.2, and from the reference detector 7.r. Thanks to the two measuring detectors 7.1, 7.2, the control unit 8 is able to determine the concentration of two different target gases in the gas sample Gp. With the help of the signal of the reference detector 7.r, the control unit 8 is able to computationally compensate to a certain extent for the influence of environmental conditions, for example of water droplets and particles, in the measuring chamber 20. Ideally, the reference detector 7.r does not react to a target gas in the measuring chamber 20, while ideally each measuring detector 7.1, 7.2 and the reference detector 7.r react equally to environmental conditions.

The gas-measuring device 100 can comprise, in addition to the photoelectric sensor or instead of the photoelectric sensor, a sensor that operates according to a different measuring principle. For example, the sensor comprises a detector and a compensator, both of which are heated. The detector oxidizes combustible target gas in the measuring chamber 20. The oxidation of the target gas releases heat energy, and the released heat energy is measured. The released heat energy correlates with the target gas concentration sought. Ideally, the compensator responds to environmental conditions in the same way as the detector but oxidizes flammable target gas to a lesser extent or even not at all. Such a sensor has become known under the name “heat tone sensor.” Another name is “catalytic sensor” because the detector contains a catalytic material that allows or at least enhances oxidation. In one implementation the detector and the compensator each are implemented as a pellistor.

The sensor can also be implemented as an electrochemical sensor and comprise a measuring electrode, a counter electrode, an ionically conductive electrolyte between the two electrodes, and preferably a reference electrode. This sensor works on the principle of a fuel cell with the target gas as the fuel. An electric current flows between the measuring electrode and the counter electrode. The electrical charge is an indicator for the target gas concentration in the measuring chamber 20 and is measured.

The sensor can also be a photoacoustic or photopneumatic sensor. The electromagnetic radiation triggers an acoustic or pneumatic effect in the measuring chamber and/or in a reference chamber. A target gas to be detected attenuates the electromagnetic radiation and thereby influences the acoustic or pneumatic effect. The acoustic or pneumatic effect is an indicator for the target gas concentrations sought.

It is also possible for the gas-measuring device 100 to comprise at least two sensors, wherein the two sensors preferably operate according to two different measuring principles. This further increases the reliability that a target gas to be detected is actually detected and creates redundancy.

Unless expressly stated otherwise, the following description relates to the embodiment according to FIG. 1, to the embodiments according to FIGS. 2 to 4, and to the embodiments according to FIG. 5. The same reference signs have the same meanings.

A display and operating unit 10 is inserted into a wall of the housing 1. The display and operating unit 10 comprises two indicator lights 11.1, 11.2, a selection switch 12, and two display units 13.1 and 13.2. The respective concentration of the two target gases in the gas sample Gp is displayed on the two display units 13.1 and 13.2. The display unit 13.1 relates to the concentration of a first target gas, and the display unit 13.2 relates to the concentration of a second target gas. The first column is assigned to a measuring position Mp.1, and the second column is assigned to a measuring position Mp.2. These two measuring positions Mp.1 and Mp.2 are spatially distanced from one another and are explained below.

A first fluid-conveying unit 2.1 and a second fluid-conveying unit 2.2 are arranged inside the housing 1. In the indicated implementation, both fluid-conveying units 2.1 and 2.2 have the form of a pump; in other possible implementations, they have the form of a piston-cylinder unit or a blower or a fan. A fluid-guiding unit 14.1 in the form of a hose or a tube connects an output of the first pump 2.1 to a first inlet into the measuring chamber 20. A fluid-guiding unit 14.2 in the shape of a hose or a tube connects an output of the second pump 21 to a second inlet into the measuring chamber 20. Of course, it is also possible for the two outputs of the two pumps 2.1 and 2.2 to be connected to the same inlet into the measuring chamber 20.

A first connector 16.1 and a second connector 16.2 are inserted into the housing 1. A fluid-guiding unit 9.1 in the form of a hose or a tube connects the first connector 16.1 to an input of the first pump 9.1. A fluid-guiding unit 9.2 in the shape of a hose or a tube connects the second connector 16.2 to an input of the second pump 9.2. A hose 3.1 is attached to the first connector 16.1 in a fluid-tight and detachable manner. The free (remote) end of the hose 3.1 is connected to a gas inlet 4.1. A hose 3.2 is attached to the second connector 16.2 fluid-tightly and detachably. The free end of the hose 3.2 is connected to a gas inlet 4.2. The two hoses 3.1 and 3.2 as well as the two gas inlets 4.1 and 4.2 also belong to the gas-measuring device 100 and are located outside the housing 1.

The two gas inlets 4.1 and 4.2 can be positioned independently of one another at different measuring positions. The measuring position of the first gas inlet 4.1 is designated Mp.1, the measuring position of the second gas inlet 4.2 is designated Mp.2. In the application shown, the hose 3.2 is guided (passed) through an opening Ö in a wall of a tank Ta. The gas inlet 4.2 is located inside the tank Ta. Thanks to the hose 3.2, it is not necessary to position the gas-measuring device 100 inside the tank Ta. The gas-measuring device 100 is thereby protected from environmental influences that may occur inside the tank Ta. The gas inlet 4.1 is located outdoors and outside the tank Ta.

In the embodiments according to FIG. 1 to FIG. 4, the first gas inlet 4.1 functions as the only element of a first gas inlet set, the second gas inlet 4.2 functions as the only element of a second gas inlet set. Hereinafter, the first gas inlet set is designated GM.1, the second gas inlet set is designated GM.2. In the embodiment according to FIG. 5a, a further gas inlet 4.3 additionally belongs to the first gas inlet set GM.1.

In the embodiment according to FIG. 1, the first pump 2.1 at least temporarily sucks in a gas sample Gp through
the gas inlet 4.1 outside the housing 1 and the container Ta,
the hose 3.1,
the connector 16.1 and
the hose 9.1
   and conveys the gas sample Gp through the hose 14.1 into the measuring chamber 20. The second pump 2.1 at least temporarily sucks in a gas sample Gp through the gas inlet 4.2 in the tank Ta, the hose 3.2, the connector 16.2 and the hose 9.2 and conveys the gas sample Gp through the hose 14.2 into the measuring chamber 20. Arrows indicate the flow direction of the gas. In the other embodiments described below, a gas sample Gp also enters the measuring chamber 20.

If at least one pump 2.1, 2.2 is switched on and conveys a gas sample Gp into the measuring chamber 20, preferably a positive pressure temporarily develops in the measuring chamber 20. The measuring chamber 20 is connected to an outlet Aus in the housing 1 by a line 18. The positive pressure in the measuring chamber 20 is released through the line 18 and the outlet Aus into the environment of the gas-measuring device 100.

Preferably, the measuring chamber 20 is flushed out in a previous step, i.e. cleaned of an old gas sample. In one implementation, the old gas sample is conveyed into the environment through a connector 16.1, 16.2. In another implementation, the old gas sample is conveyed into the environment through the line 18 and the outlet Aus.

Note: In the example shown, the gas-measuring device 100 comprises two pumps 2.1, 2.2 and two gas inlets 4.1, 4.2. It is also possible that the gas-measuring device 100 comprises n pumps and at least n gas inlets, where n>=3.

In the exemplary embodiment, the control unit 8 is able to automatically actuate each pump 2.1, 2.2 independently of the other or any other pump 2.2, 2.1 and in particular to switch it on and off, but this is not absolutely necessary.

The control unit 8 belongs to the actuation arrangement of the exemplary embodiment. In the exemplary embodiment, the control unit 8 is arranged inside the housing 1. The control unit 8 can also be positioned spatially remotely from the gas-measuring device 100.

Different modes in which the control unit 8 does this are possible and are described below.

According to the disclosure, the control unit 8 is able to switch the gas-measuring device 100 back and forth between a detection mode and a differentiation mode. These two modes are first described with reference to FIG. 1. In addition to these two modes, there is usually also an idle state in which all pumps 2.1, 2.2 are switched off.

In the detection mode, all pumps 2.1, 2.2 are switched on. The gas sample Gp in the measuring chamber 20 is therefore created by mixing the gas samples that are sucked-in through the gas inlets 4.1 and 4.2. The control unit 8 checks whether the concentration of at least one target gas or the sum of the target gas concentrations in the measuring chamber 20 is above a specified lower concentration limit. The concentration limit can be the same for each target gas to be detected or can vary from target gas to target gas.

If the control unit 8 has detected the event in which the concentration of at least one target gas is above the respective lower concentration limit, the control unit 8 automatically switches the gas-measuring device 100 from the detection mode to the differentiation mode. In the differentiation mode, the or each measuring position Mp.1, Mp.2 from which the excessive target gas concentration originates is detected. In the differentiation mode, each gas inlet 4.1, 4.2 and thus each producible fluid connection is assigned a respective pump 2.1, 2.2, and one measuring phase is carried out for each gas inlet 4.1, 4.2 and thus for each producible fluid connection.

In the first embodiment, which is shown in FIG. 1, the first pump 2.1 is assigned to the first gas inlet 4.1 and thus to the first gas inlet set GM.1, and the second pump 2.2 is assigned to the second gas inlet 4.2 and thus to the second gas inlet set GM.2. The measuring phase that is carried out for the gas inlet set 2.x (x=1, 2, . . . ) is described below.

In this measuring phase, the assigned pump 2.x is in a switched-on state or is switched on, and preferably the other or any other pump 2.y (y #x) is in a switched-off state or is switched off or at least put into an energy-saving mode. It is possible for the measuring phases to follow one another directly. It is also possible for an evaluation phase to occur between two consecutive measuring phases, in which evaluation phase all pumps 2.1, 2.2 are switched off.

The measuring phase that is assigned to the gas inlet set GM.x and in which the pump 2.x is switched on is so long that in this measuring phase the measuring chamber 20 is flushed out and filled with a new gas sample Gp. At the end of the measuring phase, the measuring chamber 20 contains a gas sample Gp that was sucked-in exclusively by the pump 2.x and thus comes from the gas inlet or gas inlets of the gas inlet set GM.x to which this pump 2.x is assigned, wherein the gas inlets of the gas inlet set GM.x are fluid-connected to this pump 2.x. In many cases, a positive pressure in the measuring chamber 20 prevents gas from another gas inlet from entering the measuring chamber 20.

The radiation source 5 emits electromagnetic radiation eS into the measuring chamber 20, and the reflected radiation eS strikes the detector component 7. At the end of the measuring phase or in the subsequent evaluation phase, the control unit 8 evaluates the signals from the detector component 7. These signals relate to the gas sample Gp that comes from the gas inlet set GM.x, in the example shown from the gas inlet 4.x.

After each measuring phase had been completed at least once, a respective gas sample Gp from each gas inlet set GM.x was in the measuring chamber 20. Preferably, the measuring chamber 20 was flushed between two measuring phases. At the latest after completion of the measuring phases, the control unit 8 determines, for the or each target gas to be detected, the respective concentration and the measuring position Mp.1, Mp.2 at which this target gas occurs. This is possible because, in the example shown, each gas inlet set GM.x consists only of exactly one gas inlet 4.x and, at the end of the measuring phase for the gas inlet 4.x, the measuring chamber 20 is filled with a gas sample Gp exclusively from this gas inlet 4.x. If a gas inlet set comprises at least two gas inlets, at least the gas inlet set from which an increased target gas concentration originates is known after the end of the measuring phases.

The control unit 8 causes the display and operating unit 10 to output a corresponding message. In the example shown, the display unit 13.1 indicates that the first target gas occurs at a concentration of 0.1 at the measuring position Mp.1 and at a concentration of 0.2 at the measuring position Mp.2. The display unit 13.2 indicates that the first target gas occurs at a concentration of 10.1 at the measuring position Mp.1 and at a concentration of 1.0 at the measuring position Mp.2.

Preferably, the pumps 2.1, 2.2 are operated in an energy-saving mode when the gas-measuring device 100 is in the detection mode. In the energy-saving mode, the pumps 2.1 and 2.2 achieve a lower volume flow than in the differentiation mode. This embodiment saves electrical energy, which is in particular important if or while the gas-measuring device 100 is not connected to a stationary power supply network and therefore comprises its own power supply unit.

Preferably, a user can use the selection switch 12 to select between at least two of the following alternatives:
The gas-measuring device 100 is initially operated in a detection mode. If the gas-measuring device 100 has detected in the detection mode that at least one target gas is present with an increased target gas concentration, the gas-measuring device 100 automatically switches to the differentiation mode and carries out the measuring phases described above.
The gas-measuring device 100 is operated in the differentiation mode or the detection mode depending on the position of the selector switch.

A second embodiment of the gas-measuring device 100 is described below with reference to FIGS. 2 to 4. Again, the gas-measuring device 100 can be operated in the detection mode and automatically be switched to the differentiation mode when at least one target gas is present at an increased target gas concentration. The following describes how the second embodiment differs from the first embodiment.

In the first embodiment, preferably each pump 2.1, 2.2 is able to achieve the same volume flow. Each pump 2.1, 2.2 can be switched on and off independently of the or any other pump. In the second embodiment, the second pump 2.2 is preferably able to achieve a lower volume flow and/or mass flow than the first pump 2.1. As a result, the second pump 2.2 consumes less electrical energy than the first pump 2.1. The lower volume flow is indicated in FIG. 2 by a smaller symbol for the second pump 2.2. In the second embodiment, it is also possible, but not absolutely necessary, for each pump 2.1, 2.2 to be switched on and off independently of the or every other pump.

Between the connectors 16.1 and 16.2 on the one hand and the pumps 2.1 and 2.2 on the other hand, a changeover valve 30 is arranged, which is also located inside the housing 1. In the exemplary embodiment, the changeover valve 30 comprises two inputs E.1 and E.2 as well as two outputs A.1 and A.2. The first input E.1 is connected to the first connector 16.1 via the line 9.1, the second input E.2 is connected to the second connector 16.2 via the line 9.2. As a result, the first input E.1 is in a fluid connection with the first gas inlet 4.1, and the second input E.2 is in a fluid connection with the second gas inlet 4.2. The first output A.1 is connected to the first pump 2.1 via a line 31.1, the second output A.2 is connected to the second pump 2.2 via a line 31.2. The outputs A.1 and A.2 are thus in a respective fluid connection with the measuring chamber 20.

A possible first implementation of the changeover valve 30 is described below with reference to FIG. 3, and a second possible implementation is described with reference to FIG. 4. FIG. 3 shows the three possible positions of the changeover valve 30 according to the first implementation, FIG. 4 shows two of the three possible positions according to the second implementation.

In contrast to the first embodiment, in the second embodiment not every producible fluid connection between a gas inlet 4.x and the measuring chamber 20 is actually established. Rather, a producible fluid connection is actually established by moving the changeover valve 30 into a corresponding position. Therefore, the measuring phases differ from each other by different positions of the changeover valve 30 in the second embodiment.

In both implementations, in the detection mode the second pump 2.2 is switched on and the first pump 2.1 is switched off. The changeover valve 30 according to the first implementation is in the position shown in FIG. 3a. This position is the third position. A fluid connection is established between the lines 9.1 and 9.2 on the one hand and the line 31.2 on the other hand and leads through the changeover valve 30—more precisely: from the inputs E.1 and E.2 to the second output A.2. The switched-on second pump 2.2 sucks in gas from the two gas inlets 4.1 and 4.2. The gas sample Gp in the measuring chamber 20 is created by mixing the gases sucked-in from the two gas inlets 4.1 and 4.2. The line 31.1 is disconnected from the gas inlets 4.1 and 4.2.

Just as in the first embodiment, the control unit 8 switches the gas-measuring device 100 to the differentiation mode if the control unit 8 has detected, during operation in the detection mode, that at least one target gas occurs at an increased target gas concentration. During operation in the differentiation mode, the second pump 2.2 is switched off and the first pump 2.1 is switched on.

In the second embodiment, a fluid-conveying unit is also assigned to each gas inlet set 2.x and therefore each gas inlet 4.x during the measuring phase. In the second embodiment, however, in contrast to the first embodiment, the same first pump 2.1 is assigned to each gas inlet set 2.x and therefore to each gas inlet 4.x, i.e. to the pump with the larger volume flow. The first pump 2.1 is therefore assigned to both the first gas inlet 4.1 and the second gas inlet 4.2.

In the second embodiment, the first pump 2.1 preferably remains permanently switched on in the differentiation mode. The second pump 2.2 is preferably switched off in the differentiation mode or at least switched to an energy-saving mode in order to save electrical energy.

FIG. 3b relates to the measuring phase that is assigned to the first gas inlet set GM.1 and thus to the first gas inlet 4.1. A fluid connection is established between the first gas inlet 4.1 and the switched-on first pump 2.1 and leads through the line 3.1, the connector 16.1, the line 9.1, the changeover valve 30 in the position of FIG. 3b, and the line 31.1. This position is the first position. The changeover valve 30 blocks a fluid connection between the second gas inlet 4.2 and the measuring chamber 20. For this purpose, the changeover valve 30 connects the first input E.1 to the first output A.1 and disconnects the first input E.1 from the second output A.2 and the second input E.2 from the two outputs A.1 and A.2. At the end of this measuring phase, the measuring chamber 20 is filled with a gas sample Gp that comes exclusively from the gas inlet 4.1.

Accordingly, FIG. 3c relates to the measuring phase that is assigned to the second gas inlet set GM.2 and thus to the second gas inlet 4.2. In this drawing, the changeover valve is in the second position. The statements in the previous paragraph also apply correspondingly to the second gas inlet 4.2.

FIG. 4 shows the second implementation of the changeover valve 30.

The changeover valve 30 comprises three individual changeover valves 30.1, 30.2, 30.3. The two individual changeover valves 30.1 and 30.2 are connected in parallel and each comprise one input and two outputs. The input of the individual changeover valve 30.1 is connected to the first input E.1 of the changeover valve 30, and the input of the individual changeover valve 30.2 is connected to the second input E.2. Both an output of the individual changeover valve 30.1 and an output of the individual changeover valve 30.2 are connected to the first output A. 1 of the changeover valve 30. The two other outputs of the individual changeover valves 30.1 and 30.2 are connected to the two inputs of the third individual changeover valve 30.3 which is arranged downstream. The output of the individual changeover valve 30.3 is connected to the second output A.2.

When the gas-measuring device 100 is operated in the detection mode, the changeover valve 30 is in the third position, and FIG. 4a shows the third position according to the second implementation. Gas from the first gas inlet 4.1 flows through the first input E.1 and the individual changeover valve 30.1 to the first output A.1. Gas from the second gas inlet 4.2 flows through the second input E.2 and the individual changeover valve 30.2 also to the first output A.1. In the detection mode, the position of the individual changeover valve 30.3 is irrelevant. As in the first implementation, the second pump 2.2 is switched on and conveys gas through the line 31.2, and the first pump 2.1 is preferably switched off.

When the gas-measuring device carries out the measuring phase for the first gas inlet set GM.1 (gas inlet 4.1), the changeover valve 30 is in the first position, and FIG. 4b shows the first position. Gas from the first input E.1 flows first through the individual changeover valve 30.1 and then through the individual changeover valve 30.3 to the second output A.2. The position of the individual changeover valve 30.2 is irrelevant. As in the first implementation, the first pump 2.1 is switched on and conveys gas through the line 31.1, and the second pump 2.2 is preferably switched off.

FIG. 5 schematically shows two different possible uses of the gas-measuring device 100 according to the disclosure. Even with these different possible uses, the gas-measuring device 100 can be operated in a detection mode and in a differentiation mode, both in accordance with the first embodiment and in accordance with the second embodiment.

In FIG. 5a, the first gas inlet 4.1 and a third gas inlet 4.3 are connected to the same hose 3.1. This hose 3.1 is in turn attached to the first connector 16.1 in a fluid-tight and detachable manner. It is possible for gas from the first measuring position Mp.1 to flow through the first gas inlet 4.1 and for gas from the third measuring position Mp.3 to flow through the third gas inlet 4.3 and through a fluid connection to the hose 3.1 into the measuring chamber 20. In the measuring chamber 20, a mixture of gas from the first measuring position Mp.1 and gas from the third measuring position Mp.3 is then formed as the gas sample Gp. With this embodiment, both the first measuring position Mp.1 and the third measuring position Mp.3 can be monitored. The same measuring phase is carried out for both the first measuring position Mp.1 and the third measuring position Mp.3. Of course, the gas-measuring device 100 is not able to distinguish whether an increased target gas concentration originates from the first measuring position Mp.1 or from the third measuring position Mp.3. In the example of FIG. 5a, the two gas inlets 4.1 and 4.3 function as a first gas inlet set GM.1, and the gas inlet 4.2 functions as a second gas inlet set GM.2.

In FIG. 5b, no hose is attached to the first connector 16.1, and an opening of the first connector 16.1 functions as a gas inlet 4.4. The gas inlet 4.4 in the first connector 16.1 functions as the first gas inlet set GM.1. A fourth measuring position Mp.4 therefore functions as a measuring position directly in front of the first connector 16.1.

FIG. 6 illustrates the detection mode and the measuring phases using a flow chart. In this example, the sensor 5, 6, 7 is able to measure both the concentration of a first target gas and the concentration of a second target gas in a gas sample Gp located in the measuring chamber 20. The flow chart applies to the embodiment according to FIG. 1 and, after a corresponding slight modification, also to the other embodiments. In the flow chart of FIG. 6, the reference signs mean the following:

| | |
|---|---|
| S1 | The gas-measuring device 100 is switched on. |
| S2 | The gas-measuring device 100 is operated in the detection mode. |
| | In the measuring chamber 20, a gas sample Gp is created, which |
| | is formed by mixing the gases from all gas inlets 4.1, 4.2, 4.3, |
| | 4.4. |
| S3 | The sensor 5, 6, 7 measures the concentration of the first target |
| | gas in the gas sample Gp. |
| S4 | The sensor 5, 6, 7 measures the concentration of the second |
| | target gas in the gas sample Gp. |
| con1 | the measured concentration of the first target gas in the gas |
| | sample Gp, wherein the gas sample Gp is a mixture of gases |
| | from all gas inlets 4.1, 4.2 |
| con2 | the measured concentration of the second target gas in the gas |
| | sample Gp, wherein the gas sample Gp is a mixture of gases |
| | from all gas inlets 4.1, 4.2 |
| con1 > | It is checked whether the first target gas concentration is greater |
| uS1? | than the lower concentration limit uS1 specified for the first |
| | target gas |
| con2 > | It is checked whether the second target gas concentration is |
| uS2? | greater than the lower concentration limit uS2 specified for the |
| | second target gas |
| S5 | The control unit 8 switches the gas-measuring device 100 to the |
| | differentiation mode. |
| S6 | The measuring phase for the first gas inlet 4.1 is carried out. In |
| | the first embodiment, the first pump 2.1 is in a switched-on state |
| | or is switched on and the second pump 2.2 is in a switched-off |
| | state or is switched off. In the second embodiment, the |
| | changeover valve 30 establishes a fluid connection between the |
| | first gas inlet 4.1 and the measuring chamber 20 and interrupts |
| | the fluid connection between the second gas inlet 4.2 and the |
| | measuring chamber 20. |
| con1.1 | the measured concentration of the first target gas in the gas |
| | sample Gp, wherein the gas sample Gp comes exclusively from |
| | the first gas inlet 4.1 |
| con1.2 | the measured concentration of the second target gas in the gas |
| | sample Gp, wherein the gas sample Gp comes only from the first |
| | gas inlet 4.1 |
| S7 | The measuring phase for the second gas inlet 4.2 is carried out. |
| con2.1 | the measured concentration of the first target gas in the gas |
| | sample Gp, wherein the gas sample Gp comes only from the |
| | second gas inlet 4.2 |
| con2.2 | the measured concentration of the second target gas in the gas |
| | sample Gp, wherein the gas sample Gp comes only from the |
| | second gas inlet 4.2 |
| S8 | For each measuring position Mp.1 and Mp.2, the two display |
| | units 13.1 and 13.2 show the respective concentration of the |
| | two target gases at these two measuring positions |

LIST OF REFERENCE SIGNS

| | |
|---|---|
| 1 | Housing of the gas-measuring device 100, accommodates in its |
| | interior the pumps 2.1 and 2.2, the measuring chamber 20, the |
| | hoses 9.1, 9.2, 14.1 and 14.2, the optional changeover valve 30, |
| | and the control unit 8, and supports the display and operating |
| | unit 10 |
| 2.1 | First pump, connected to the hoses 9.1 and 14.1 |
| 2.2 | Second pump, connected to the hoses 9.2 and 14.2 |
| 3.1 | First hose, leads from the first gas inlet 4.1 to the first connector |
| | 16.1 |
| 3.2 | Second hose, leads from the second gas inlet 4.2 to the second |
| | connector 16.2 |
| 4.1 | First gas inlet, located at the free end of the hose 3.1, belongs to |
| | the first gas inlet set GM.1 |
| 4.2 | Second gas inlet, located at the free end of the hose 3.2, belongs |
| | to the second gas inlet set GM.2 |
| 4.3 | Further gas inlet, located at the free end of the hose 3.1, belongs |
| | to the first gas inlet set GM.1 |
| 4.4 | Further gas inlet, located in the first connector 16.1, belongs to |
| | the first gas inlet set GM.1 |
| 5 | Infrared radiation source, emits the electromagnetic radiation eS |
| | into the measuring chamber 20 |
| 6 | Mirror on a wall of the measuring chamber 20, reflects |
| | electromagnetic radiation eS |
| 7 | Detector component, comprises the first measuring detector 7.1, |
| | the second measuring detector 7.2, and a reference detector 7.r |
| 7.1 | First measuring detector of the detector component 7 |
| 7.2 | Second measuring detector of the detector component 7 |
| 7.r | Reference detector of the detector component 7 |
| 8 | Signal-processing control unit, receives and processes signals |
| | from the detector component 7, actuates the pumps 2.1 and 2.2 |
| | as well as the changeover valve 30 |
| 9.1 | Hose from the first connector 16.1 to the first pump 2.1, located |
| | inside the housing 1 |
| 9.2 | Hose from the second connector 16.2 to the second pump 2.2, |
| | arranged inside the housing 1 |
| 10 | Display and operating unit in the housing 1, comprises the |
| | indicator lights 11.1 and 11.2, the selection switch 12 and the |
| | display units 13.1 and 13.2 |
| 11.1, | Indicator light of the display and operating unit 10 |
| 11.2 | |
| 12 | Selection switch of the display and operating unit 10 |
| 13.1 | First display unit of the display and operating unit 10, displays |
| | the concentration of a first target gas at each measuring position |
| | Mp.1, Mp.2 |
| 13.2 | Second display unit of the display and operating unit 10, |
| | displays the concentration of a second target gas at each |
| | measuring position Mp.1, Mp.2 |
| 14.1 | Hose from the first pump 2.1 to the measuring chamber 20 |
| 14.2 | Hose from the second pump 2.2 to the measuring chamber 20 |
| 16.1 | First connector on the housing 1, connects the hoses 3.1 and 9.1 |
| | to one another |
| 16.2 | Second connector on the housing 1, connects the hoses 3.2 and |
| | 9.2 to one another |
| 18 | Hose from the measuring chamber 20 to the outlet Aus |
| 20 | Measuring chamber, accommodates in its interior the gas sample |
| | Gp and, on two walls, the radiation source 5, the detector |
| | component 7 and the mirror 6 |
| 30 | Changeover valve, has the inputs E.1 and E.2 and the outputs |
| | A.1 and A.2, has three possible positions, is actuated by the |
| | control unit 8 |
| 30.1, | Individual changeover valve according to the second |
| 30.2, | implementation of the changeover valve 30 |
| 30.3 | |
| 31.1 | Line from the changeover valve 30 to the first pump 2.1 |
| 31.2 | Line from the changeover valve 30 to the second pump 2.2 |
| 100 | Gas-measuring device, comprises the housing 1, the control unit |
| | 8, the measuring chamber 20, the radiation source 5, the detector |
| | component 7, the mirror 6, the two pumps 2.1 and 2.2, the hoses |
| | 9.1, 9.2, 14.1, 14.2 inside the housing 1, the connectors 16.1 and |
| | 16.2, the outlet Aus, the hoses 3.1 and 3.2 outside on the |
| | connectors 16.1 and 16.2, the optional changeover valve 30 and |
| | the gas inlets 4.1 and 4.2 on the hoses 3.1 and 3.2, and the |
| | display and operating unit 10 |
| Aus | Outlet in the housing 1, connected to the measuring chamber 20 |
| | by the hose 18 |
| A.1 | First output of the changeover valve 30, connected to the |
| | measuring chamber 20 via the line 31.1 |
| A.2 | Second output of the changeover valve 30, connected to the |
| | measuring chamber 20 via the line 31.2 |
| E.1 | First input of the changeover valve 30, connected via the line 9.1 |
| | to the connector 16.1 |
| E.2 | Second input of the changeover valve 30, connected via the line |
| | 9.2 to the connector 16.2 |
| eS | Electromagnetic radiation, is emitted by the radiation source 5, |
| | penetrates the gas sample Gp once, is reflected by the mirror 6, |
| | penetrates the gas sample Gp again and strikes the detector |
| | component 7 |
| GM.1 | First gas inlet set, comprises the gas inlet 4.1 or the gas inlet 4.4, |
| | in one embodiment additionally the gas inlet 4.3 |
| GM.2 | Second gas inlet set, comprises the gas inlet 4.2 |
| Gp | Gas sample in the measuring chamber 20 |
| Mp.1 | First measuring position, is assigned to the first gas inlet 4.1 |
| Mp.2 | Second measuring position, is assigned to the second gas inlet |
| | 4.2 |
| Mp.3 | Third measuring position, is assigned to the fourth gas inlet 4.3 |
| Mp.4 | Fourth measuring position, is assigned to the fourth gas inlet 4.4 |
| Ö | Opening in the tank Ta through which the second hose 3.2 leads |
| Ta | Tank with the opening Ö, accommodates the gas inlet 4.2 |

1-16. (canceled)

17. An actuation arrangement for actuating a gas-measuring device:
wherein the gas-measuring device comprises:
a measuring chamber;
a sensor;
at least two gas inlet sets;
a fluid connection arrangement; and
at least one fluid-conveying unit;
wherein each gas inlet set comprises at least one respective gas inlet;
wherein the measuring chamber is configured to hold a gas sample;
wherein the sensor is configured to measure, while the gas sample is in the measuring chamber, one or more of:
a concentration of at least one target gas in a gas sample; or
a sum of several target gas concentrations;
wherein the fluid connection arrangement is configured to connect the or each gas inlet, of each gas inlet set, to the measuring chamber by a respective fluid connection;
wherein the at least one fluid-conveying unit is configured to convey, from the at least one respective gas inlet of at least one gas inlet set, a respective quantity of a gas through the fluid connection arrangement to the measuring chamber;
wherein the actuation arrangement is configured to actuate the gas-measuring device such that the gas-measuring device:
is operated, depending on the actuation:
either in a detection mode,
or in a differentiation mode; and
when being operated in the differentiation mode, performs, for at least one gas inlet set of the at least two gas inlet sets, at least one respective measuring phase;
wherein the actuation arrangement is further configured to actuate the gas-measuring device such that, during operation in the detection mode:
the respective fluid connection between the or every gas inlet, of each gas inlet set, and the measuring chamber is established; and
the at least one fluid-conveying unit conveys, from the or every gas inlet, of each gas inlet set, the respective quantity of the gas into the measuring chamber; and
wherein the actuation arrangement is further configured to actuate the gas-measuring device such that, when the at least one respective measuring phase for a given gas inlet set is performed:
the respective fluid connection between the or each gas inlet of this gas inlet set and the measuring chamber is established;
a flow of gas from each gas inlet not belonging to this gas inlet set into the measuring chamber is prevented; and
the at least one fluid-conveying unit conveys, from the or each gas inlet of this gas inlet set, the respective quantity of the gas into the measuring chamber.

18. The actuation arrangement of claim 17:
wherein the at least one fluid-conveying unit comprises a first fluid-conveying unit and a second fluid-conveying unit;
wherein to each gas inlet set a respective fluid-conveying unit of the first fluid-conveying unit and the second fluid-conveying unit is assigned;
wherein the respective fluid-conveying unit assigned to a given gas inlet set is configured to convey the respective quantity of gas from each gas inlet of this gas inlet set through the fluid connection arrangement to the measuring chamber;
wherein the actuation arrangement is configured to switch each fluid-conveying unit on and off independently of the other fluid-conveying unit; and
wherein the actuation arrangement is configured to actuate the gas-measuring device such that:
during operation in the detection mode, the first and second fluid-conveying units are switched on; and
while the respective measuring phase for a given gas inlet set is performed:
the fluid-conveying unit being assigned to this given gas inlet set is switched on; and
the other fluid-conveying unit is switched off.

19. The actuation arrangement of claim 17:
wherein the gas-measuring device comprises a controllable changeover valve with a first input and a second input and at least one output;
wherein the fluid connection arrangement comprises:
a first input fluid-guiding unit;
a second input fluid-guiding unit; and
an output fluid-guiding unit;
wherein the first input fluid-guiding unit establishes a fluid connection between the first input and each gas inlet of a first gas inlet set;
wherein the second input fluid-guiding unit establishes a fluid connection between the second input and each gas inlet of a second gas inlet set different from the first gas inlet set;
wherein the output fluid-guiding unit establishes a fluid connection between the output and the measuring chamber;
wherein the actuation arrangement is configured to actuate the changeover valve such that:
depending on the actuation, the changeover valve is moved into a first position, a second position, or a third position;
wherein the changeover valve in the first position establishes a fluid connection between the first input and the output and disconnects the second input from the output;
wherein the changeover valve in the second position establishes a fluid connection between the second input and the output and disconnects the first input from the output; and
wherein the changeover valve in the third position establishes a fluid connection between the first input, the second input, and the output; and
wherein the actuation arrangement is configured to actuate the changeover valve such that the changeover valve:
is, during the detection mode, in the third position;
is, during a measuring phase for the first gas inlet set, in the first position; and
is, during a measuring phase for the second gas inlet set, in the second position.

20. The actuation arrangement of claim 19:
wherein the output of the changeover valve is a first output, and the changeover valve comprises a second output;
wherein the output fluid-guiding unit is a first output fluid-guiding unit and the fluid connection arrangement comprises a second output fluid-guiding unit:
wherein the first output fluid-guiding unit establishes a fluid connection between the first output and the measuring chamber; and
wherein the second output fluid-guiding unit establishes a fluid connection between the second output and the measuring chamber;
wherein the at least one fluid-conveying unit comprises a first fluid-conveying unit and a second fluid-conveying unit;
wherein the first fluid-conveying unit is configured to convey a gas through the first output fluid-guiding unit to the measuring chamber;
wherein the second fluid-conveying unit is configured to convey a gas through the second output fluid-guiding unit to the measuring chamber; and
wherein the changeover valve:
establishes, in the first position, a fluid connection between the first input and the first output and disconnects the second input from the first and second outputs;
establishes, in the second position, a fluid connection between the second input and the first output and disconnects the first input from the first and second outputs; and
establishes, in the third position, a fluid connection between the first input, the second input, and the second output.

21. The actuation arrangement of claim 20, wherein:
the actuation arrangement is configured to actuate the two fluid-conveying units such that:
the first fluid-conveying unit is switched on in the differentiation mode and switched off in the detection mode; and
the second fluid-conveying unit is switched on in the detection mode and switched off in the differentiation mode.

22. The actuation arrangement of claim 20, wherein:
the first fluid-conveying unit is configured to achieve, relative to the second fluid-conveying unit, one or more of:
a greater volume flow;
a greater mass flow; or
a greater consumption of electrical energy.

23. The actuation arrangement of claim 20, wherein the actuation arrangement is configured to actuate the gas-measuring device such that:
the gas-measuring device is in the detection mode as long as a measured concentration of the or each target gas in the gas sample or a measured sum of target gas concentrations in the measuring chamber is within a given concentration range; and
the gas-measuring device switches into the differentiation mode when the measured concentration of the or at least one target gas in the gas sample or the measured sum of target gas concentrations in the measuring chamber is outside the respective concentration range.

24. A gas-measuring device comprising:
a measuring chamber;
a sensor;
at least two gas inlet sets;
a fluid connection arrangement; and
at least one fluid-conveying unit;
wherein each gas inlet set comprises at least one respective gas inlet;
wherein the measuring chamber is configured to hold a gas sample;
wherein the sensor is configured to measure, while the gas sample is in the measuring chamber, one or more of:
a concentration of at least one target gas in the gas sample, or
a sum of two or more target gas concentrations;
wherein the fluid connection arrangement is configured to connect the or each gas inlet, of each gas inlet set, to the measuring chamber by a respective fluid connection;
wherein the at least one fluid-conveying unit is configured to convey, from the at least one respective gas inlet, of at least one gas inlet set, a respective quantity of a gas through the fluid connection arrangement to the measuring chamber;
wherein the gas-measuring device is configured to be operated:
either in a detection mode,
or in a differentiation mode;
wherein the gas-measuring device is further configured to, during operation in the differentiation mode, perform, for at least one gas inlet set of the at least two gas inlet sets, at least one respective measuring phase;
wherein the gas-measuring device is further configured such that, during operation in the detection mode:
the respective fluid connection between the or every gas inlet of each gas inlet set, and the measuring chamber is established; and
the at least one fluid-conveying unit conveys, from the at least one respective gas inlet, of each gas inlet set, the respective quantity of the gas into the measuring chamber; and
wherein the gas-measuring device is further configured such that, when the at least one respective measuring phase for a given gas inlet set is performed:
the respective fluid connection between each gas inlet of this gas inlet set and the measuring chamber is established;
a flow of gas, from each gas inlet not belonging to this gas inlet set, into the measuring chamber is prevented; and
the at least one fluid-conveying unit conveys, from each gas inlet of this gas inlet set, the respective quantity of the gas into the measuring chamber.

25. The gas-measuring device of claim 24, further comprising:
a housing;
wherein the sensor and the measuring chamber are located within the housing;
wherein each gas inlet of a first gas inlet set and each gas inlet of a second gas inlet set are located outside the housing;
wherein each gas inlet of the first gas inlet set is configured to be positioned freely relative to the housing and relative to each gas inlet of the second gas inlet set within a range predefined by the fluid connection arrangement; and
wherein each gas inlet of the second gas inlet set is configured to be positioned freely relative to the housing and relative to each gas inlet of the first gas inlet set within a range predefined by the fluid connection arrangement.

26. The gas-measuring device of claim 24:
wherein the gas-measuring device comprises a controllable changeover valve with a first input and a second input and at least one output;
wherein the fluid connection arrangement comprises:
a first input fluid-guiding unit;
a second input fluid-guiding unit; and
an output fluid-guiding unit;
wherein the first input fluid-guiding unit establishes a fluid connection between the first input and the or each gas inlet of a first gas inlet set;
wherein the second input fluid-guiding unit establishes a fluid connection between the second input and the or each gas inlet of a second gas inlet set different from the first gas inlet set;
wherein the output fluid-guiding unit establishes a respective fluid connection between the output and the measuring chamber;
wherein the changeover valve in a first position establishes a fluid connection between the first input and the output and disconnects the second input from the output;
wherein the changeover valve in a second position establishes a fluid connection between the second input and the output and disconnects the first input from the output;
wherein the changeover valve in a third position establishes a fluid connection between the first input and the second input on the one hand and the output on the other hand; and
wherein the gas-measuring device is configured such that the changeover valve:
is, when being in the detection mode, in the third position;
is, during the measuring phase for the first gas inlet set, in the first position; and
is, during the measuring phase for the second gas inlet set, in the second position.

27. The gas-measuring device of claim 26, further comprising:
a signal-processing control unit,
wherein the control unit is configured to cause the gas-measuring device to transition between the detection mode and the differentiation mode by means of a corresponding actuation of the changeover valve.

28. The gas-measuring device of claim 27, wherein the control unit is configured to:
cause the gas-measuring device to be in the detection mode as long as a measured concentration of each target gas in the gas sample or a measured sum of target gas concentrations in the measuring chamber is within a given concentration range; and
switch the gas-measuring device to the differentiation mode when the measured concentration of a given target gas in the gas sample or the measured sum of target gas concentrations in the measuring chamber is outside the concentration range.

29. An operating method, comprising:
with a gas-measuring device comprising:
a measuring chamber;
a sensor;
at least two gas inlet sets;
a fluid connection arrangement; and
at least one fluid-conveying unit;
wherein each gas inlet set comprises at least one respective gas inlet; and
wherein the fluid connection arrangement is configured to connect each gas inlet, of each gas inlet set, to the measuring chamber,
the operating method comprises the steps of:
operating the gas-measuring device at times in a detection mode and at times in a differentiation mode;
performing, during operation in the differentiation mode, for at least one gas inlet set, at least one respective measuring phase;
wherein, in either mode, steps are carried out in which:
a gas sample is conveyed through the fluid connection arrangement to the measuring chamber;
the measuring chamber holds the gas sample; and
the sensor measures a concentration of at least one target gas in the gas sample or a sum of several target gas concentrations while the gas sample is in the measuring chamber;
during operation in the detection mode:
establishing a respective fluid connection between each gas inlet of each gas inlet set and the measuring chamber; and
conveying, using the at least one fluid-conveying unit, from the or every gas inlet of each gas inlet set a respective quantity of a gas into the measuring chamber; and
wherein, when the at least one respective measuring phase for a given gas inlet set is performed:
the respective fluid connection is established between each gas inlet of this gas inlet set and the measuring chamber;
a flow of gas, from each gas inlet not belonging to this gas inlet set, into the measuring chamber is prevented; and
the at least one fluid-conveying unit conveys, from each gas inlet of this gas inlet set, a respective quantity of the gas into the measuring chamber.

30. The operating method of claim 29, wherein:
the gas-measuring device is operated in the detection mode as long as a measured concentration of each target gas in the gas sample or a measured sum of target gas concentrations in the measuring chamber is within a specified concentration range; and
the gas-measuring device is switched to the differentiation mode when the measured concentration of a given target gas in the gas sample or the measured sum of target gas concentrations in the measuring chamber is outside the respective concentration range.

31. The operating method of claim 29, wherein the at least one fluid-conveying unit comprises a first fluid-conveying unit and a second fluid-conveying unit, the method further comprising:
switching the first fluid-conveying unit on in the differentiation mode and switching the first fluid-conveying unit off in the detection mode; and
switching the second fluid-conveying unit on in the detection mode and switching the second fluid-conveying unit off in the differentiation mode.

32. The operating method of claim 31, wherein the first fluid-conveying unit is configured to achieve, relative to the second fluid-conveying unit, one or more of:
a greater volume flow;
a greater mass flow; or
a greater consumption of electrical energy.

33. A measuring method for measuring a concentration of at least one target gas, the method being performed with a gas-measuring device comprising:
a measuring chamber;
a sensor;
at least two gas inlet sets;
a fluid connection arrangement; and
at least one fluid-conveying unit;
wherein each gas inlet set comprises at least one respective gas inlet;
wherein the fluid connection arrangement is configured to connect each gas inlet, of each gas inlet set, to the measuring chamber by a respective fluid connection,
the method comprising the steps of:
operating the gas-measuring device:
either in a detection mode,
or in a differentiation mode;
wherein, in either mode, steps are carried-out in which:
a gas sample through the fluid connection arrangement into the measuring chamber;
the measuring chamber holds the gas sample; and
the sensor measures a concentration of at least one of:
at least one target gas in the gas sample, or
a sum of several target gas concentrations while the gas sample is in the measuring chamber;
during operation in the detection mode:
establishing a respective fluid connection between each gas inlet of each gas inlet set and the measuring chamber; and
conveying, using the at least one fluid-conveying unit, from the or every gas inlet, of each gas inlet set, a respective quantity of a gas into the measuring chamber; and
performing, during operation in the differentiation mode, for at least one gas inlet set, at least one respective measuring phase;
wherein, while the at least one respective measuring phase for a given gas inlet set is performed:
the respective fluid connection is established between each gas inlet of this gas inlet set and the measuring chamber;
a flow of gas from each gas inlet not belonging to this gas inlet set into the measuring chamber is prevented; and
the at least one fluid-conveying unit conveys, from each gas inlet of this gas inlet set, a respective quantity of the gas into the measuring chamber.

34. The measuring method of claim 33, wherein:
the gas-measuring device is operated in the detection mode as long as a measured concentration of the or each target gas in the gas sample or a measured sum of target gas concentrations in the measuring chamber is within a specified concentration range; and
a detection of an event that the measured concentration of at least one target gas in the gas sample or the measured sum of the target gas concentrations in the measuring chamber is outside the respective concentration range causes the gas-measuring device to be operated in the differentiation mode.

35. The measuring method of claim 34, wherein:
a detection of an event that the measured concentration of the or every target gas in the gas sample or the measured sum of target gas concentrations in the measuring chamber is again within the respective concentration range causes the gas-measuring device to be operated again in the detection mode.

36. The measuring method of claim 33, wherein the at least one fluid-conveying unit comprises a first fluid-conveying unit and a second fluid-conveying unit, the method further comprising:
switching the first fluid-conveying unit on in the differentiation mode and switching the first fluid-conveying unit off in the detection mode; and
switching the second fluid-conveying unit on in the detection mode and switching the second fluid-conveying unit off in the differentiation mode.

37. The measuring method of claim 33, wherein the first fluid-conveying unit is configured to achieve, relative to the second fluid-conveying unit, one or more of:
a greater volume flow;
a greater mass flow; or
a greater consumption of electrical energy.
